(19) United States
(12) Patent Application Publication
(10) Pub. No.: US 2021/0030951 A1
(43) Pub. Date: Feb. 4, 2021

(54) Infusion Set Having Reduced Patient Pain

(71) Applicants: Leif Bowman, San Diego, CA (US); Nicholas John Fowley, Edinburgh (GB); Mark H. Scott, San Diego, CA (US)

(72) Inventors: Leif Bowman, San Diego, CA (US); Nicholas John Fowley, Edinburgh (GB); Mark H. Scott, San Diego, CA (US)

(21) Appl. No.: 16/853,316

(22) Filed: Apr. 20, 2020

Related U.S. Application Data
(60) Provisional application No. 62/836,634, filed on Apr. 20, 2019.

(51) Int. Cl. A61M 5/158; A61M 5/142

(52) U.S. Cl. CPC A61M 5/158; A61M 5/14248; A61M 2005/14252; A61M 2202/048; A61M 2202/0225; A61M 2202/0266; A61M 2202/0484; A61M 2205/3606

(57) ABSTRACT

A painless infusion set has an affixed portion and may have a disposable or reusable insertion device portion. The infusion set or its inserter may have methods or mechanisms to utilize gas, liquid, gel, or solid within phase or through phase change with user-controlled variably slow insertion to reduce pain. In one example, a canister of a pain reducing material is in the insertion device. The pain reducing material can be dispensed onto the skin before, during, or after insertion of the needle or cannula to reduce pain.

### RELATED APPLICATIONS

This application claims priority to U.S. provisional application No. 62/836,634, filed Apr. 20, 2019, and entitled “An Insertion Set Having Reduced Patient Pain,” which is incorporated herein in its entirety. This application is related to U.S. patent application Ser. No. 16/714,701, filed Dec. 14, 2019 and entitled “Kinkless Infusion Set for Medical use,” and to U.S. patent application Ser. No. 16/560,315, filed Sep. 4, 2019 and entitled “Extended Use Infusion Set for Medical Use,” both of which are incorporated herein as if set forth in their entirety.

### FIELD OF THE INVENTION

This invention relates to the needle, cannula, or cannula and inserter needle portion of an infusion set, and in particular infusion sets and sensors, as they relate to medicine delivery, biological fluid gathering, and biological sensing.

### BACKGROUND OF THE INVENTION

A large number of persons in the US and around the world suffer from diseases like diabetes. Such persons achieve treatment through fluid medicines delivered either with injections or through infusion sets.

Infusion sets typically have a duration of 1-3 days before it is necessary to remove the infusion set and find a new location on the body for another infusion set. The result is patients needing between 100+ to 300+ disposable infusion sets annually.

Patients who use do not use infusion sets typically obtain their insulin through multiple daily injections (MDI). These patients have even more frequent and possibly painful injections than those utilizing infusion sets with insulin pumps.

Patients using continuous glucose monitoring (CGM) to measure their glucose levels will change their sensors typically every 3-6 days, although some may not need to change for 12 days. The result is an additional approximately 20-120 injections annually for CGMs with an insertion that is similar to an injection.

The current state of the art for infusion sets uses an introducer needle with a soft cannula around it for the length of the needle that penetrates the skin. The end of the soft cannula is tipped/formed onto the surface of the introducer needle to ensure a tight fit that can withstand the forces of insertion through the skin.

As the introducer needle has a beveled edge to create a sharp point/edge to penetrate the skin, it is necessary for the introducer needle to protrude approximately 3 mm beyond the end of the soft cannula. The result is that a patient who requires a soft cannula tip to deliver medication into the body at a depth of 6 mm must insert 9 mm deep. This creates a deeper wound than necessary and is additionally intimidating to the user.

Further, the introducer needles used in the current infusion sets are seen by the user upon removal, and this view of the needle is intimidating to the user and reinforces the negative aspect of inserting infusion sets.

### SUMMARY OF THE DISCLOSURE

It is an object of the present invention to provide an improved infusion set and/or sensor set which reduces or removes the issues mentioned.

Embodiments of the present invention are directed to an infusion set with the capability to have a slow insertion.

A preferred embodiment is directed to an infusion set with the capability to have a controlled slow insertion.

A preferred embodiment is directed to an infusion set with the capability to have a controlled slow insertion in which the timing and speed may be varied by the user.

A preferred embodiment is directed to an infusion set with the capability to have a controlled slow insertion in which speed may be varied by the user, but the timing is not known precisely by the user.

A preferred embodiment is directed to an infusion set which the skin may be stretched in one or more directions during insertion.

A preferred embodiment is directed to an infusion set in which a liquid or solid may cool, warm, or numb the skin during insertion.

A preferred embodiment is directed to an infusion set in which a liquid or solid may undergo phase change in order to cool, warm, or numb the skin during insertion.

A preferred embodiment is directed to an infusion set with the capability to have a controlled slow insertion to an effective depth and position of a sensor such as for a continuous glucose monitor (CGM) for the purposes of interaction with biological processes can provide useful data.

A preferred embodiment is directed to an infusion set with the capability to have an insertion in which the skin is stretched in one or more directions and the insertion is to an effective depth and position of a sensor such as for a continuous glucose monitor (CGM) for the purposes of interaction with biological processes can provide useful data.

A preferred embodiment is directed to an infusion set with the capability to have an insertion in which a solid, liquid, gel, or gas cools, warms, or numbs the skin before, during, or after insertion to an effective depth and position of a sensor such as for a continuous glucose monitor (CGM) for the purposes of interaction with biological processes can provide useful data.

### BRIEF DESCRIPTION OF THE DRAWINGS

FIG. 1 is an illustration showing an exemplary embodiment of the present invention with a user-controlled slow insertion.

FIG. 2 is an illustration showing an exemplary embodiment of the present invention with a user-controlled variably slow insertion.

FIG. 3 illustrates an infusion set with a biological sensor, like might be found in a continuous glucose monitor with a user-controlled insertion.

FIG. 4 is an illustration showing an exemplary embodiment of the present invention with a user-controlled slow insertion to a therapeutic depth variably set by the user.

FIG. 5A is an illustration showing an exemplary embodiment of the present invention which stretches the skin in one or more directions during insertion.

FIG. 5B is an illustration showing an exemplary embodiment of the present invention which stretches the skin in one or more directions during insertion, partially actuated.

FIG. 6 is an illustration showing an exemplary embodiment of the infusion set with a solid, liquid, gel, or gas that cools, warms or numbs the skin before, during, or after insertion.

FIG. 7 is an illustration showing an example of the infusion set in which a solid, liquid or gel undergoes one or more phase changes to cool, warm, or numb the skin before, during, or after insertion.

FIG. 8 is an illustration showing an example of the present invention in which a gas flows through an expansion nozzle to cool, warm or numb the skin before, during, or after insertion.

FIG. 9 is an illustration showing the present invention in which an expendable solid, gel, liquid, or gas element cools, warms or numbs the skin before during, or after insertion.

FIG. 10 is an illustration showing an exemplary embodiment in which a solid, gel, liquid, or gas cools, warms, or numbs the skin before, during, or after insertion, with multiple ports in the infusion set for different insertion points within the same site with a straight, curved, or helical kinkless cannula being removed from one insertion port and a different kinkless cannula being inserted, straight or angled, at another insertion port.

FIG. 11 is an illustration showing an exemplary embodiment in which a solid, gel, liquid, or gas cools, warms, or numbs the skin before, during, or after insertion, with multiple ports in the infusion set for different insertion points within the same site with a sensor inserted in one insertion point and a straight, curved, or helical kinkless cannula inserted, straight or angled, at another insertion port.

FIG. 12 shows an exemplary embodiment with multiple ports in the infusion set to permit relocation of the sensor and/or straight, curved, or helical needle in the same infusion set at the same site, in order to achieve new depots through new insertions.

FIG. 13 is an illustration showing an exemplary embodiment in which a solid, gel, liquid, or gas cools, warms, or numbs the skin before, during, or after insertion, of a cannula on an infusion set with multiple ports and a biological sensor.

FIG. 14 is an illustration showing an exemplary embodiment of the present invention prior to actuation of the gel application in which a numbing and/or disinfecting gel is applied before, during or after the inserter needle and cannula to cool, warm, disinfect or numb the skin before, during, or after insertion.

FIGS. 15A, 15B and 15C have illustration views showing an exemplary embodiment of the extended use infusion set with variable user-controlled manual insertion to reduce or eliminate pain in accordance with the present invention.

### DETAILED DESCRIPTION OF THE EXEMPLARY EMBODIMENTS

The embodiments and examples shown here are to provide enough information to fully understand the invention. One skilled in the art will understand how minor changes or deviations can be made and still be within the scope of the invention. The following description of exemplary embodiments of the invention is not intended to limit the scope of the invention to these exemplary embodiments, but rather to enable any person skilled in the art to make and use the invention. To assist in a clear and unambiguous understanding of the disclosure, the following definitions are used:

Definitions

A bolus is generally a single dose of insulin, typically a larger dose administered at once and often at mealtime. The purpose is to provide additional insulin to help the body address the carbohydrate intake of the meal. A bolus may also be given if there are other foreseen or unforeseen events affecting blood sugar. A bolus may be administered via an infusion set which also delivers basal insulin, or may be injected separately.

The basal insulin level is the amount of insulin referred to as necessary to address the diabetic patient's need for insulin between meals or other foreseen or unforeseen events, such as mealtime or snacks. The basal insulin dosage is often a lower level, delivered more regularly or nearly continuously, between meals.

A cannula is similar to a hollow metal needle, but often made of a softer, flexible material which can bend. Some cannula designs incorporate an introducer needle, which may or may not be hollow, to help the cannula achieve its desired position.

An introducer needle is a hollow or solid needle which generally does not deliver medicine itself, but instead helps the cannula penetrate the skin and achieve its desired position to deliver medicine for therapeutic benefit at a subdermal or subcutaneous depth and position.

A needle is a sharp object which is used to penetrate the skin and push through the body to a desired depth or position. A needle may be hollow or solid. It may allow medicine to travel through it into the body, or it may act only as an introducer needle for a softer and/or more flexible cannula and be retracted after its positioning function, or it may perform both medicine delivery and introducer functions.

A depot is a location where medicine is delivered by the needle or cannula, and generally the “depth and location” within the patient tissue where the medicine is absorbed by the body.

An insertion device is a part of an infusion set which may be used to assist the attachment of the affixed portion of the infusion set, and the insertion of the cannula and inserter needle, and may be discarded or reused after actuation. Methods and mechanisms described herein to reduce or eliminate pain entirely may be in the affixed portion of the infusion set, or may be resident in the insertion device, or some combination.

As shown in the included figures, the illustrations depict instances of infusion sets inserted into the skin for the purposes of delivering fluid medicine into the tissue beneath the outer layer of skin, such as insulin for the treatment of diabetes subcutaneously. However it will be understood that the invention may also be utilized for delivery of other medicine, hormones, vitamins, saline, including fluids containing dissolved or suspended solids if in the future such a treatment is created. The invention may be used for the placement of sensors capable of measuring biological information, such as glucose levels, salinity, red or white blood cells, T-cell counts, dissolved oxygen, ketones, lactate, or the like on a continuous or intermittent basis, whether for information, entertainment, or compliance purposes only, as part of a feedback loop in medicine delivery, or to aid in a combination of manual and automated administration of fluid described above, whether that manual administration is through the infusion set or administered elsewhere on the body.

FIG. 1 shows an infusion set 100 with a curved cannula 101, which may be slowly inserted by the user to a depth at which one or more therapeutic doses of medicine may be delivered. The adhesive pad is 110, the head is 120, and the inserter mechanism is 135. The skin is 170. The skin layers are (top to bottom) epidermis, dermis, subcutaneous tissue, muscle.

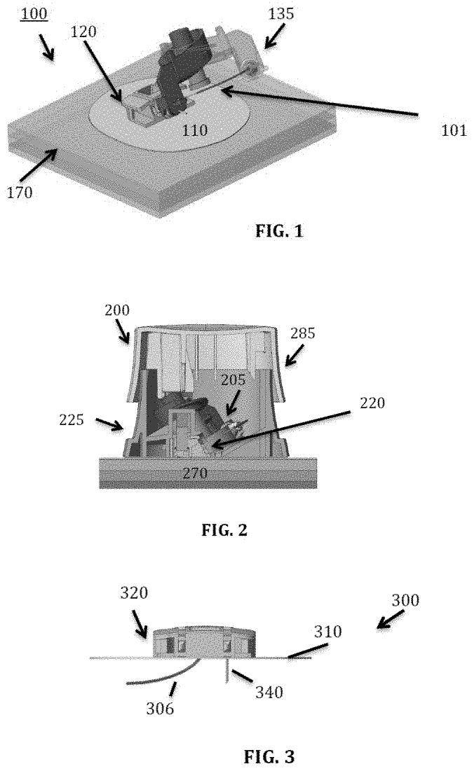

FIG. 2 shows an infusion set 200 with a curved kinkless cannula 205, which may be slowly inserted with variable speed by the user to a depth at which one or therapeutic doses of medicine may be delivered. The head is 220. Button 285 is in its initial position, prior to actuation of the inserter. The base is part 225. The 270 skin layers are (top to bottom) epidermis, dermis, subcutaneous tissue, muscle.

FIG. 3 shows an infusion set 300 with a biological sensor 340, which may be slowly inserted to an effective depth for sensing biological activity or levels that may be utilized for entertainment or information purposes only, or may also be used for the wearer or others to inform or control manually administered medicine at the site or elsewhere, or be used by devices attached to the same or another infusion set to provide intermittent or continuous fluid delivery. The cannula is 306, the adhesive pad is 310 and the head is 320.

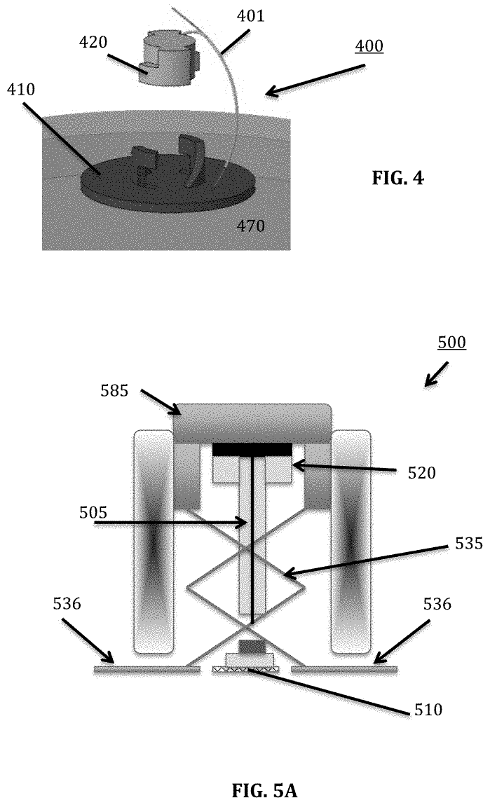

FIG. 4 shows a helical needle 401 on infusion set 400 which may slowly be inserted to a therapeutic depth variably set by the user. The adhesive pad is 410, the head is 420. The skin is 470.

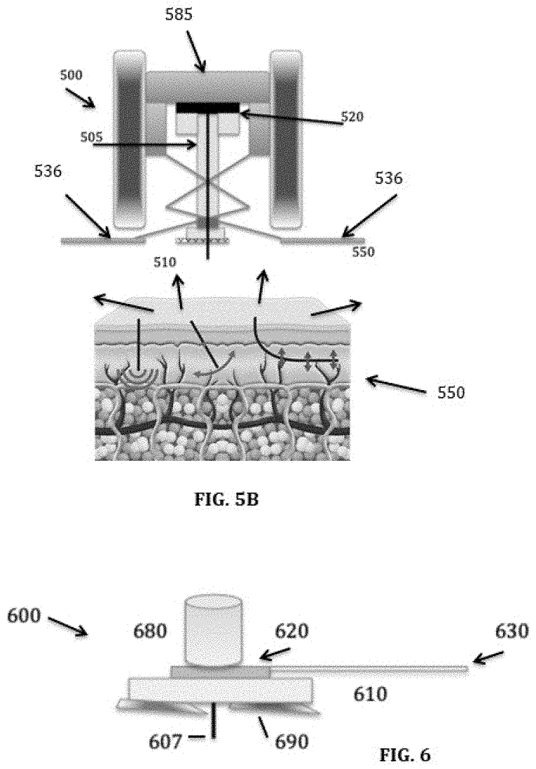

FIG. 5A shows an infusion set 500 with a straight cannula 505 in which the skin may be stretched in one or more directions during insertion. The adhesive pad is 510, the head is 520. As illustrated, a stretching mechanism 535 is in the head 520, which may be in the form of a mechanical linkage mechanism, coil, spring or lever. It will be appreciated that other mechanical stretching devices may be used. The stretching mechanical linkage mechanism 535 is operably connected to stretch pads 536, which are intended to engage the surface of the skin when the insertion set is place against the patient's skin 570. Is some cases, the pads 536 may have light adhesive, or alternative, may simply frictionally engage the skin. When first placed on the skin, the stretching mechanism 535 is relaxed, and the stretch pads 536 are relatively close together and rest on the skin. In one example, the sketch pads 536 are in the form of a circle pad with a concentrically formed opening through which the cannula will be inserted.

In operation, the infusion set 500 is brought in contact with the patient's skin 550, with then button 585 or other actuator disengaged and in an initial position. The patient or caregiver begins pressing the button 585 into the head 520, which places the stretching mechanism 535 under tension. As the button is further pressed, the stretching mechanism causes the stretching pads 536 to spread apart, thereby pulling and extending the skin between the pads, or within the concentric area surrounded by the pads. FIG. 5B shows an infusion set 500 with a straight cannula 505 in which the skin is being stretched in one or more directions during insertion. The adhesive pad is 510 and the head is 520. After the skin has been stretch with the stretch pads 536, the patient continues to press button 585 into head 520, which begins inserting the cannula 505 into and under the skin. As the skin is stretched and under tension, the patient will feel reduce pain as compared to insertion in an unstretched area of skin.

It will be appreciated that the stretching pads may be arranged to contact the skin before the adhesive pad is applied. In this way, the button can cause the activation mechanism to move the stretching pads apart and stretch the skin prior to the application of the adhesive pad. It will also be understood that the stretching pads may stretch the skin in one direction, multiple directions, linearly, or in a circular motion. Further, although the infusion set is described with the pads stretching the skin, the infusion set may be modified to cause the skin be bunched together.

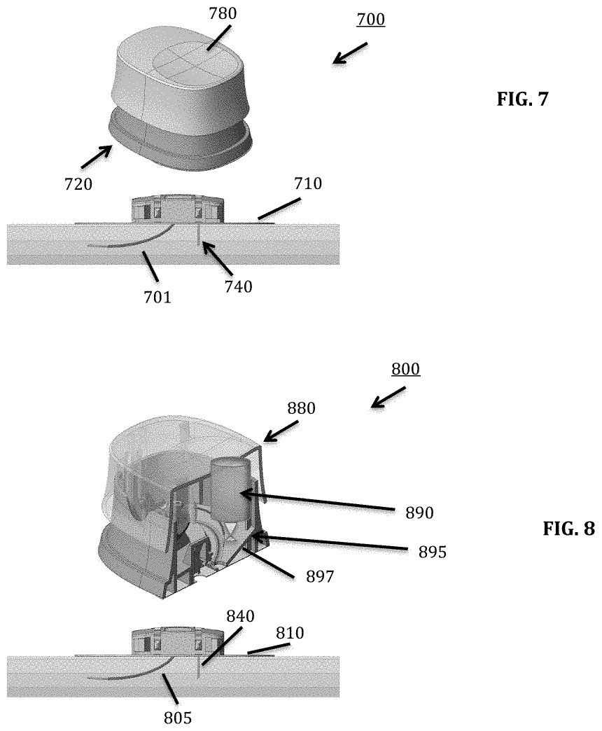

FIG. 6 shows an exemplary embodiment of the infusion set 600 in which a solid, liquid, gel or gas cools, warms, or numbs the skin before, during, or after needle insertion. The adhesive pad is 610, the head is 620, and the tubing to the infusion pump is 630. The perpendicular cannula, needle, cannula with inserter needle or cannula with repositioning needle is 607. The insertion device used for initial insertion or introducer needle removal is 680. The cooling material is 690.

FIG. 7 shows an exemplary embodiment of the infusion set 700 in which a solid, liquid, gel, or gas undergoes one or more phase changes to cool, warm, or numb the skin before, during, or after insertion with a helical cannula 701 with an inserter needle and an insertion device 780 that may be used at initial placement, subsequent introducer needle removal, or in all of these cases. This insertion device may be common with the one utilized in FIG. 6, but with a curved needle, or may be of different construction. The adhesive pad is 710, the head is 720, and the biological sensor is 740.

FIG. 8 shows an exemplary embodiment of the infusion set 800 with a gas that flows through an expansion nozzle and conducts through a heat transfer material to cool, warm, or numb the skin before, during, or after insertion of a curved cannula 805 and an inserter needle and insertion device 880 that may be used at initial placement, subsequent introducer needle removal, or in all of these cases. This insertion device may be common with the one utilized with a curved needle in FIG. 6, or may be of different construction. The adhesive pad is 810, the head is 820, and the biological sensor is 840. The inserter 880 contains gas cartridge 890 and the nozzle is 895. The heat transfer material is 897.

FIG. 9 shows an exemplary embodiment of the infusion set 900 with inserter 980 that contains dispenser 950 which dispenses an expendable solid, liquid, gel, or gas 960 that cools, warms, or numbs the skin before, during, or after insertion of a helical needle 901 and a biological sensor 940 both residing on the same infusion set 900. The wearer may take advantage of the increased duration of the infusion set and spacing afforded by the needle and sensor geometries. The adhesive pad is 910 and the head is 920.

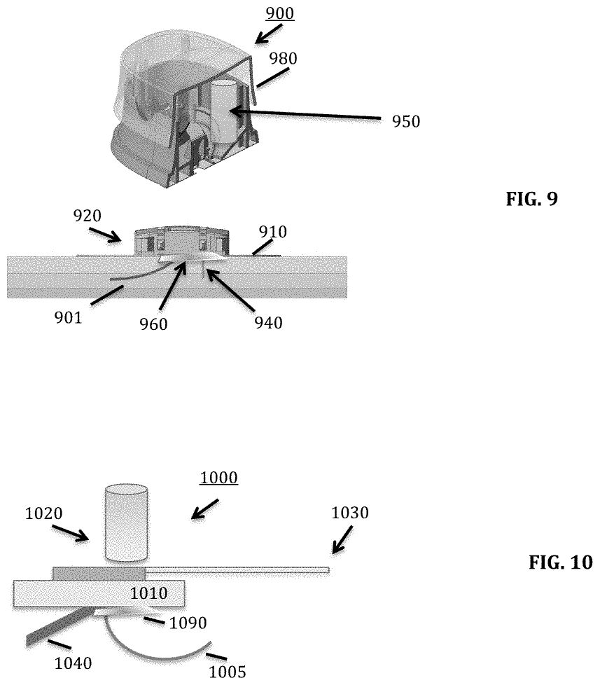

FIG. 10 shows an exemplary embodiment of the infusion set 1000 in which a solid, liquid, gel, or gas cools, warms, or numbs the skin before, during, or after insertion of a kinkless curved needle 1005 with a biological sensor 1040, like might be found with a continuous glucose monitor. The wearer may prefer having both sensor and medicine delivery on the same infusion set due to reduction in complexity and longer time between site reuse, since only one site is used at a time. The adhesive pad is 1010, the head is 1020, and the tubing to the infusion pump is 1030. The cooling material is 1090.

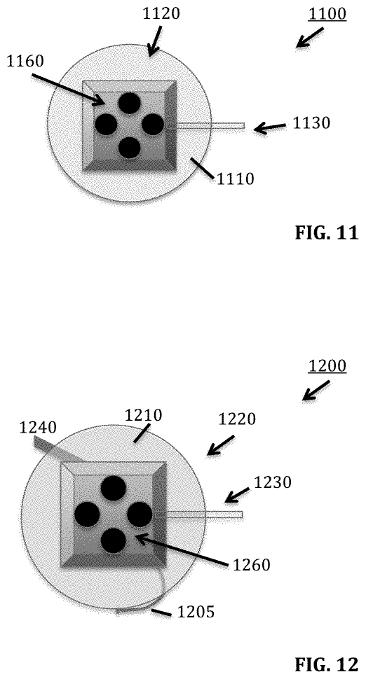

FIG. 11 shows an exemplary embodiment of the infusion set 1100 in which a solid, gel, liquid, or gas cools, warms, or numbs the skin before, during, or after insertion, with multiple ports 1160 in the infusion set to permit relocation of the straight, curved or helical needle in the same infusion set at the same site, in order to achieve new depots through new insertions. The adhesive pad is 1110, the head is 1120, and the tubing to the infusion pump is 1130.

FIG. 12 shows an exemplary embodiment in which a solid, gel, liquid, or gas cools, warms, or numbs the skin before, during, or after insertion, with a multiple ports 1260 in the infusion set 1200 to permit relocation of the sensor 1240 and/or straight, curved, or helical needle 1205 in the same infusion set at the same site, in order to achieve new depots through new insertions. It may be necessary or desired to relocate the sensor, or further insert or partially remove the sensor to sense different biological processes or other reasons. It may be necessary or desired to relocate the needle, cannula, or cannula with inserter needle to another port to achieve a new depot for increased therapeutic delivery over the original depot. The adhesive pad is 1210, the head is 1220, and the tubing to the infusion pump is 1230. Advantageously, the same hole may be reused, in some cases. Advantageously, some constructions of cannula and/or sensor may permit two or more items to be inserted through the same port.

One skilled in the art will understand that a gas may be Carbon Dioxide or Nitrogen, for examples, that a liquid may be hydrogen peroxide, alcohol, or iodine, for examples, a gel may be Neosporin, for example, and a solid may be ice, for example. The gases, liquids, gels, and solids used as examples are easily imagined and familiar, although the actual materials used may similarly be of elemental nature, such as CO2, N2, and ice or, commercially available, such as hydrogen peroxide formulations, or Neosporin brand medicines, or of proprietary formulations, such as a mixed gas or a heat-transfer solid with a formulation, mixture, or material combination that is a trade secret. These may be selected for their heating, cooling, sanitizing, numbing, or heat transfer capabilities, or some combination of these properties.

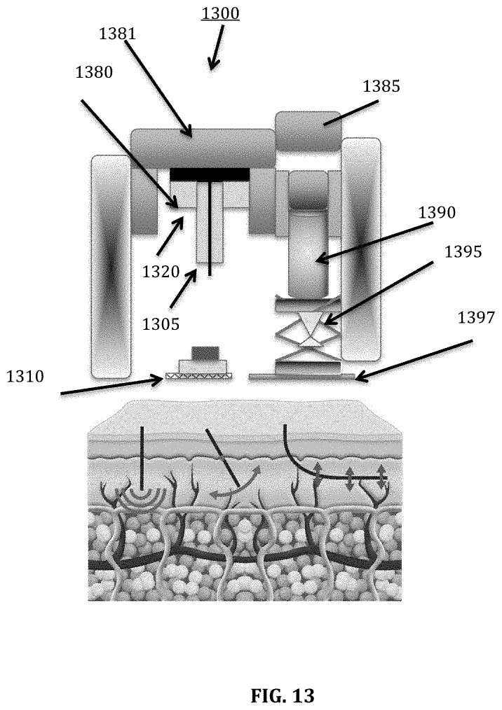

FIG. 13 shows an exemplary embodiment in which pressing button 1385 discharges a gas that flows through an expansion nozzle and conducts through a heat transfer material to cool, warm, or numb the skin before, during, or after insertion of a cannula 1305 and an inserter needle and insertion device 1380 actuated by pushing the second button 1381 that may be used at initial placement, subsequent introducer needle removal, or in all of these cases. This insertion device may be common with the one utilized with a curved needle in FIG. 6, or may be of different construction. The adhesive pad is 1310, the head is 1320. The gas cartridge is 1390 and the nozzle is 1395. The heat transfer material is 1397. The mechanical linkage mechanism shown returns the button to its initial position after actuation.

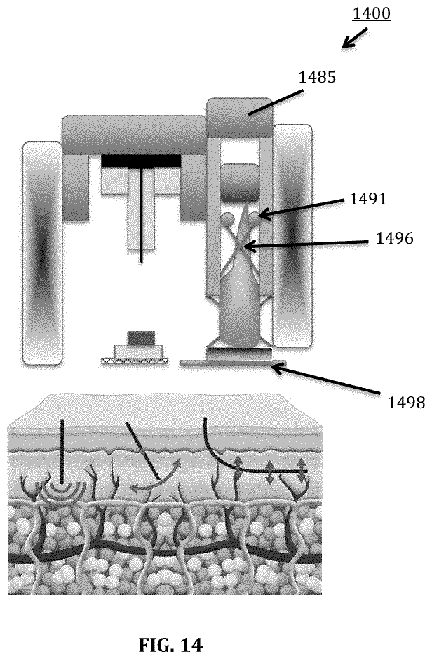

FIG. 14 shows an exemplary embodiment 1400 in which button 1485 is in its initial position, prior to actuation of the gel application. Rollers 1491 are open to allow placement of medicament tubing. Pin 1496 is at the uppermost in its travel slot, permitting the rollers to separate. One-way valve in valve plate 1498 is fully closed. During actuation of button 1485, a gel may be applied. Numbing and/or disinfecting gel is applied before, during, or after the inserter needle and cannula. Rollers 1491 have been brought together and downward by the button motion mechanical linkage mechanism and by pin 1496 sliding in its travel slot. One-way valve in valve plate 1498 is open to allow the dispense of the gel to cool, warm, disinfect, or numb the skin before, during, or after the insertion. After actuation of button 1485, rollers 1491 have been brought further downward by the button motion and by pin 1496 sliding in its travel slot. One-way valve in valve plate 1498 is closed again, after allowing the dispense of the gel to cool, warm, disinfect, or numb the skin before, during, or after the insertion.

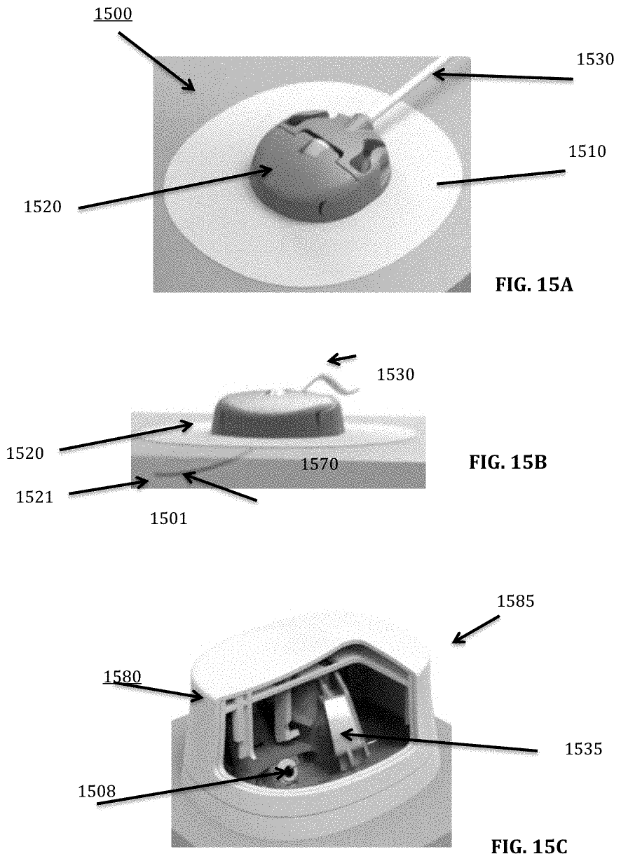

Referring now to FIG. 15A, an infusion set 1500 is illustrated. Infusion set 1500 is designed and intended to insert a cannula subcutaneously into a human at user-controlled variable-speed insertion to reduce or eliminate pain and enable a medicine or therapeutic to be delivered into the patient at a desirable depth and position. The infusion set 1500 is attached and secured to the human body using an adhesive pad 1510. A head piece 1520 is connected through tubing 1530 to the source of the medication, which in some cases may be insulin driven from an infusion pump. Example insertion mechanisms are illustrated in following sections.

Referring now to FIG. 15B, the infusion set 1500 has already been inserted under the patient's skin 1570 to an initial depth and position with a controlled insertion speed. It will be understood that a housing, mechanism, or introducer needle may have been used to insert and set the infusion set into the illustrated position. Head piece 1520 also connects to the cannula 1501. Cannula 1501 may be made from metal, plastics, or other materials appropriate for delivery of medication. It will be understood that the selection of material for cannula 1501 may be particularly selected for the type of medication to be delivered. For example, some chemotherapy medications may degrade certain materials, so materials resistant to chemical damage would need to be selected. It will also be understood that the cannula 1501 may use an introducer needle to initially place the cannula 1501 to its desired position or location 1521, with the introducer needle then being removed and discarded.

Referring now to FIG. 15C, an exemplary embodiment of the insertion device 1580 is shown after full actuation for a design in which button 1585 is fully actuated in its fully pressed position. Button 1585 is shown in its fully actuated position, as the cannula and any introducer elements have been fully inserted during controlled slow insertion travel of the actuation, and the latter portion of the travel of the button 1585 causes removal of all introducer elements, and renders the inserter ready for lifting from the body. The inserter mechanism 1535 has caused the cannula and septum module to be fully inserted and the fully removed introducer needle 1508 is visible. The button 1585 is locked down to indicate a used device and prevent secondary needle actuation.

Common and familiar methods and assemblies may not be mentioned, in order to be brief and clear. While particular preferred and alternative embodiments of the present intention have been disclosed, it will be appreciated that many various modifications and extensions of the above described technology may be implemented using the teaching of this invention. All such modifications and extensions are intended to be included within the true spirit and scope of the appended claims.

What is claimed is:

1. An infusion set having reduced insertion pain, comprising:
an affixed base portion and an insertion device;
an insertion mechanism in the insertion device and constructed to insert a cannula into the skin of a patient;
a stretching pad extending from the base portion and arrange to contact the skin of the patient;
a head portion connected to the base portion;
an actuating device in the insertion device that is engageable by the patient or caregiver, the actuating device coupled to the insertion mechanism;
a stretching mechanism in the insertion device that is coupled to the actuating device and to the stretching pads;
wherein activating the actuating device first engages the stretching mechanism to cause the stretching pad to stretch the skin of the patient, with further activation engaging the insertion mechanism to insert the cannula through the skin.

2. The infusion set according to claim 1, wherein the stretching pads cause the skin at the point of cannula insertion to be stretched and under tension.

3. The infusion set according to claim 1, wherein the insertion mechanism also includes an introducer needle within the cannula.

4. The infusion set according to claim 1, wherein the stretching mechanism is a mechanical linkage mechanism, spring, coil or lever.

5. The infusion set according to claim 1, wherein the actuating device is a lever, push button or knob.

6. The infusion set according to claim 1, wherein the stretching pads have an adhesive on the surface that contacts the patient's skin.

7. The infusion set according to claim 1, wherein the stretching pads frictionally engage the patient's skin.

8. The infusion set according to claim 1, wherein after the cannula is fully inserted, releasing the activation mechanism allows the stretching mechanism to relax and the stretching pads to allow the skin to unstretch.

9. The infusion set according to claim 1, wherein the base has an adhesive pad that attaches to the patient's skin, and the activation mechanism stretches the patent skin prior to adhesive pad engaging the patient's skin.

10. An infusion set having reduced insertion pain, comprising:
an affixed base portion and an insertion device;
an insertion mechanism in the insertion device and constructed to insert a cannula into the skin of a patient;
a head portion connected to the base portion;
an actuating device in the insertion device portion that is engageable by the patient or caregiver, the actuating device coupled to the insertion mechanism;
a canister in the insertion device for holding a pain-reducing substance;
wherein activating the actuating device dispenses the pain reducing material onto the patient's skin, and engages the insertion mechanism to insert the cannula through the skin at an area where the pain reducing material is dispensed.

11. The infusion set according to claim 10, wherein the pain-reducing material is dispensed prior to the cannula being inserted.

12. The infusion set according to claim 10, wherein the pain-reducing material is dispensed while the cannula being inserted.

13. The infusion set according to claim 10, wherein the pain-reducing material is dispensed after the cannula has been inserted.

14. The infusion set according to claim 10, wherein the pain-reducing material is in the form of a solid, liquid, gel, or gas.

15. The infusion set according to claim 10, wherein the pain-reducing material is a numbing liquid or gel.

16. The infusion set according to claim 10, wherein the pain-reducing material is a gas and the insertion device has an expansion nozzle that dispenses a cooled gas to the patient's skin.

17. The infusion set according to claim 10, wherein the pain-reducing material cools the skin.

18. The infusion set according to claim 10, wherein the insertion mechanism also includes an introduced needle within the cannula.

19. The infusion set according to claim 10, wherein the actuating device is a mechanical linkage mechanism, lever, push button or knob.

20. The infusion set according to claim 10, wherein the pain-reducing material is medicated with an anesthesia.

21. The infusion set according to claim 10, wherein the pain reducing material is an antiseptic, an anesthetic, carbon dioxide, nitrogen, hydrogen peroxide, alcohol, iodine, or ice.

22. The infusion set according to claim 10, wherein the canister is pressurized by internal pressure.

23. The infusion set according to claim 10, wherein the canister is pressurized by the activation mechanism.

24. The infusion set according to claim 10, wherein the same actuating device inserts the cannula and dispenses the pain reducing material.

25. The infusion set according to claim 10, wherein the actuating device inserts the cannula and another actuating device dispenses the pain reducing material.
